(19) United States
(12) Patent Application Publication
(10) Pub. No.: US 2026/0259169 A1
(43) Pub. Date: Sep. 3, 2026

(54) BETA-LACTAMASE SUBSTRATE COMPOUNDS AND ELECTROCHEMICAL DETECTION METHODS USING THEM

(71) Applicant: CORIS BioConcept SRL, Gembloux (BE)

(72) Inventors: Arnaud BEAUFAYS, Court-Saint-Etienne (BE); Pascal MERTENS, Haltinne (BE); Thierry LECLIPTEUX, Wépion (BE); Stéphane VINCENT, Hyon (BE)

(21) Appl. No.: 19/643,095

(22) Filed: Apr. 9, 2026

Related U.S. Application Data
(63) Continuation of application No. PCT/EP2024/078641, filed on Oct. 11, 2024.

(30) Foreign Application Priority Data
Oct. 12, 2023 (EP) 23203202.9

(51) Int. Cl. G01N 27/327; C12Q 1/04; C12Q 1/34

(52) U.S. Cl. CPC G01N 27/3277; C12Q 1/04; C12Q 1/34; G01N 2333/986

(57) ABSTRACT

A beta-lactamase substrate compound for detection of beta-lactamases, according to formula (I)

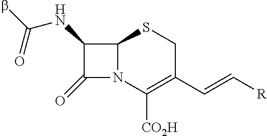

wherein β is hydrogen, C₁-C₁₆ alkyl, C₁-C₁₆ fluoroalkyl or is according to formula (II):

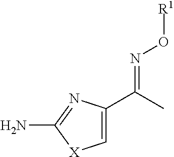

wherein X is S or O, and R¹ is C₁-C₁₆ alkyl, optionally including one or more carbonyl, hydroxyl, ether, ester or carboxyl functional groups; and R is selected from 4-cyanophenyll, 4-nitrophenyl, 4-sulfonephenyl, 2,4-dicyanophenyl, 2-nitro-4-cyanophenyl, 3,4-dinitrophenyl and 3,5-dinitrophenyl. The beta-lactamase substrate compound is hydrolyzable by a beta-lactamase; and the beta-lactamase substrate compound has a degradation at 4° C. equal to or lower than 50% after 4 weeks and equal to or lower than 70% after 18 weeks, and/or a degradation at 20° C. equal to or lower than 80% after 4 weeks and equal to or lower than 85% after 18 weeks. The degradation is determined from the difference of the integration of nuclear magnetic resonance measurement signals of the beta-lactamase substrate compound and of dimethylformamide.

### FIELD

The present disclosure is in the field of biotechnology, especially in the field of the electrochemical detection of beta-lactam antibiotics resistance. The present disclosure is related to beta-lactamase substrate compounds and electrochemical detection methods using these beta-lactamase substrate compounds.

### BACKGROUND

Beta-lactam antibiotics, which comprise penicillins, monobactams, cephalosporins (for example third-generation cephalosporins (C3Gs), cephamycins and carbapenems, are antibiotics that act by inhibiting the synthesis of peptidoglycan. Beta-lactam antibiotics are often used in first-line treatment of serious infections, both in general practice and in hospital medicine. Beta-lactam antibiotics all comprise in their structure the four atoms beta-lactam ring.

Carbapenems are a class of antibiotics that are among the strongest beta-lactam antibiotics. They are widely used in modern medicine as a robust defense against severe bacterial infections. Carbapenems exert their action by disrupting bacterial cell wall synthesis, leading to the demise of a wide spectrum of bacteria.

Their unique molecular structure grants them resistance against several bacterial enzymes that often render other antibiotics ineffective. This resilience enables carbapenems to combat both Gram-positive and Gram-negative bacteria. This renders them very valuable for treating challenging infections. Moreover, they offer a critical line of defense against multidrug-resistant organisms that do not respond to other antibiotics.

However, beta-lactam antibiotics, for example carbapenems, are not resistant against all bacterial enzymes, in particular beta-lactamases, such as carbapenemases. Beta-lactamases are enzymes produced by certain bacteria, which enable the bacteria to resist to the action of the beta-lactam antibiotics. More particularly, beta-lactamases are capable of breaking down the beta-lactam ring structure of the beta-lactam antibiotics by hydrolysis. Consequently, infections caused by these bacteria can be very difficult to treat due to the limited options available.

Therefore, a fast and accurate detection of bacteria that are resistant to beta-lactam antibiotics, and/or of the enzymes, in particular beta-lactamases, produced by these bacteria, such as extended spectrum beta-lactamases (ESBLs), hyperproduced cephalosporinases, and carbapenemases, is crucial for patient management. Such a detection contributes to the establishment of suitable antibiotic treatments.

The conventional technique used for detecting beta-lactamase-producing bacteria is based on the implementation of an antibiogram and the application of the double disc synergy test, which makes it possible to demonstrate the restoration of sensitivity to C3Gs in the presence of a penicillinase inhibitor. Despite its efficacy, implementation of this technique requires a preliminary step of culturing and isolating the bacteria, which means a delay of at least 24 hours before the production of beta-lactamases is detectable.

It is known since the 1990s that substrate compounds of beta-lactamases (which is used to indicate hydrolysable beta-lactam compounds), and the hydrolysis product obtained after hydrolysis of their beta-lactam ring by beta-lactamases have electrically active properties. Consequently, electrochemical detection thereof is possible. In particular, hydrolysis of the beta-lactam ring of a beta-lactam antibiotic by a beta-lactamase can be detected electrochemically.

The scientific publication “The Synthesis of 7-Substituted-3-dinitrostyryl Cephalosporins and Their Ability for Detecting Extended Spectrum β-Lactamases (ESBLs)”, Hanaki H. et al, The Journal of Antibiotics, 58, pp. 69-73 (2005) discloses the synthesis of 7-substituted-3-(2,4-dinitrostyryl) cephalosporin derivatives, which are nitrocefin analogs, for the detection of extended spectrum beta-lactamases (ESBLs). These cephalosporin derivatives are found to be hydrolysable by such extended spectrum beta-lactamases. Upon hydrolysis, their color changes from yellow to red, which allows the detection of ESBLs based on a color change. The developed cephalosporin derivatives do not hydrolyze when contacted with class A, C and D beta-lactamases, and thus allow not only to detect the presence of beta-lactamases in general, but also to distinguish between ESBLs and class A, C and D beta-lactamases.

An example of a cephalosporin derivative is nitrocefin, which can be hydrolyzed by all types of beta-lactamases. Another example of a cephalosporin derivative is the compound HMRZ-86, which is the E isomer of (6R,7R)-trifluoroacetate 7-[2-(2-amino-4-thiazolyl)-2-[(1-carboxy-1-methylethoxy)imino]acetyl]amino]-1-aza-3-[2-(2,4-dinitrophenyl) ethenyl]-8-oxo-5-thiabicyclo[4.2.0]oct-2-ene-2-carboxylic acid. This substrate compound is hydrolysable by the enzymes created by bacteria that are resistance to the C3Gs, i.e. producing ESBLs, hyperproduced cephalosporins and carbapenemases.

WO2016/156605 discloses that the electrochemical properties of certain beta-lactamase substrate compounds, in particular nitrocefin and HMRZ-86, are quite distinct from those of their hydrolyzed forms. This difference in electrochemical properties allows for the electrochemical detection of the presence of beta-lactamase-producing bacteria. Further, the methods disclosed allow for a shorter duration for the culture of the bacteria strains, such as a few hours, in particular from 1 to 4 hours, prior to implementing the electrochemical measurement.

However, a disadvantage of the existing detection methods includes, without being limited thereto, the fact that they can detect only a limited variety of beta-lactamases and their hydrolysis products, which limited their use and efficiency of detection, as certain beta-lactamases and the bacteria producing them may remain undetected.

A further disadvantage is that the reproducibility and stability of known detection methods is not optimal, leading to sub-efficient detection methods.

Therefore, there is a need in the field of health care to develop improved electrochemical detection methods for efficient and reliable detection of the presence of a wide range of beta-lactamases and their hydrolysis products.

### SUMMARY

The present disclosure aims to overcome one or more of the above drawbacks. The present disclosure aims to provide new beta-lactamase substrate compounds for electrochemical detection, as well as a preparation method for obtaining these compounds. In other words, it is an aim of the present disclosure to provide electrically active beta-lactam compounds which allow the electrochemical detection of beta-lactamases.

Another aim is to provide beta-lactamase substrate compounds having an improved stability, i.e. a lower degradation over time, when compared to substrates of the state of the art, in particular an improved stability during use in electrochemical detection of beta-lactamases. It is an aim of the present disclosure to provide beta-lactamase substrate compounds allowing for more efficient electrochemical detection of beta-lactamases.

Yet a further aim of the present disclosure is to provide beta-lactamase substrate compounds that are recognizable by a broader group of different beta-lactamases, i.e. which can be hydrolyzed by a broader range of beta-lactamases. This advantageously improves the discrimination of different beta-lactamases and/or the identification of bacteria producing them.

It is a further aim of the present disclosure to provide an electrochemical detection method for detecting beta-lactamases which has an improved stability and/or efficiency when compared to methods of the state of the art. It is an aim to provide such methods which have an improved reproducibility.

It is a further aim to provide a method allowing a discrimination between a broader range of beta-lactamases and the bacteria producing then when compared to methods of the state of the art, especially the beta-lactamase producing bacteria (or bacteria strains) CTXM, KPC/OXA-48, OXA-40, OXA-48, OXA-58, NDM, IMP-13, and/or VIM. Type(s)

Finally, a last aim of the present disclosure is to provide a detection method which requires a reduced time for culturing the bacterial strains, when compared to conventional detection methods, and/or which does not require isolation of the bacterial strains.

The inventors have surprisingly discovered that electrochemical detection which uses the well-known beta-lactamase substrate compound HMRZ-86 tends to lead to results which are not reproducible, thereby rendering the electrochemical detection methods using HMRZ-86 non-efficient and unstable. The inventors have further surprisingly discovered that the origin of these issues is related to the fact that HMRZ-86 is much less stable than thought in the field, and shows, in particular, a significant degradation over time when maintained at temperatures between 4° C. and 20° C.

According to a first aspect of the present disclosure, there is provided a beta-lactamase substrate compound for electrochemical detection of bacterial producing beta-lactamases as well as a preparation method for obtaining these compounds, as set out in the appended claims.

The term “beta-lactamase substrate compound” is used in the present disclosure for compounds comprising a beta-lactam ring which can be hydrolyzed by one or more beta-lactamases. In other words, a beta-lactamase substrate compound according to the present disclosure is a beta-lactam ring comprising compound that is capable of reacting with one or more beta-lactamases, in particular by hydrolysis of the beta-lactam ring by the beta-lactamase.

The beta-lactamase substrate compound according to the present disclosure is thus hydrolysable by a beta-lactamase.

The beta-lactamase substrate compound is according to formula (I)

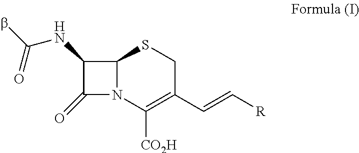

wherein
β is hydrogen, C₁-C₁₆ alkyl, C₁-C₁₆ fluoroalkyl or is according to formula (II):

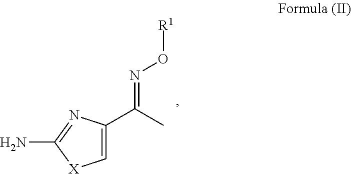

wherein
X is S or O, and
R¹ is C₁-C₁₆ alkyl, optionally comprising one or more carbonyl, hydroxyl, ether, ester or carboxyl functional groups; and
R is selected from the group consisting of 4-cyanophenyl, 4-nitrophenyl, 4-sulfonephenyl, 2,4-dicyanophenyl, 2-nitro-4-cyanophenyl, 3,4-dinitrophenyl and 3,5-dinitrophenyl.

Preferred examples of C₁-C₁₆ alkyl include C₁-C₆ alkyl, preferably methyl (CH₃) and ethyl (C₂H₅). Preferred examples of C₁-C₁₆ fluoroalkyl include C₁-C₆ fluoroalkyl, preferably C₁-C₆ perfluoroalkyl, more preferably perfluoromethyl (also named trifluoromethyl, CF₃) and perfluoroethyl (also called pentafluoroethyl, C₂F₅). The C₁-C₁₆ alkyl and C₁-C₁₆ fluoroalkyl can be linear or branched.

Advantageously, R¹ is C₁-C₆ alkyl, optionally comprising one or more carbonyl, hydroxyl, ether, ester or carboxyl functional groups. Non-limiting examples of R¹ include methyl, ethyl, n-propyl, iso-propyl, —C(CH₃)₂COOH, —CH₂COOH and —CH(CH₃)COOH.

Advantageously, β is hydrogen, methyl, perfluoromethyl or according to formula (II) with X being S and R¹ being —C(CH₃)₂COOH.

Preferred beta-lactamase substrate compounds are according to formula (I), wherein β is according to formula (II) and R is 2,4-dicyanophenyl or 2-nitro-4-cyanophenyl, preferably wherein X is S and R¹ is —C(CH₃)₂COOH.

Further preferred beta-lactamase substrate compounds are according to formula (I), wherein β is hydrogen, methyl or perfluoromethyl and R is 4-cyanophenyl.

In the present disclosure, the degradation is determined from the difference of the integration of nuclear magnetic resonance (NMR) measurement signals of the beta-lactamase substrate compound and of dimethylformamide.

Advantageously, the beta-lactamase substrate compound according to the present disclosure has a degradation at 4° C. equal to or lower than 50% after 4 weeks, preferably equal to or lower than 40%, more preferably equal to or lower than 30%, for example at most 25%, or at most 20%. Advantageously, the beta-lactamase substrate compound has a degradation at 4° C. equal to or lower than 70% after 18 weeks, preferably equal to or lower than 65%, more preferably equal to or lower than 60%, such as at most 55%.

Alternatively or additionally, and advantageously, the beta-lactamase substrate compound according to the present disclosure has a degradation at 20° C. equal to or lower than 80% after 4 weeks, preferably equal to or lower than 70%, more preferably equal to or lower than 60%, such as at most 55%, or at most 50%. Advantageously, the beta-lactamase substrate compound has a degradation at 20° C. equal to or lower than 85% after 18 weeks, preferably equal to or lower than 80%, more preferably equal to or lower than 75%, such as at most 70%, or at most 65%.

With “a degradation of x at temperature y after z weeks” is meant in the present disclosure that the beta-lactamase substrate compound shows or has a degradation of x % after maintaining the beta-lactamase substrate compound at temperature y for a duration of z weeks. In other words, the degradation testing is performed by maintaining the beta-lactamase substrate compound at a predetermined temperature (either by heating or by cooling) for a specified duration. It will be understood that the lower the percentage of degradation, the more stable a compound is.

It has been found that the beta-lactamase substrate compounds of the present disclosure show lower degradation values than prior art beta-lactamase substrate compounds, in particular HMRZ-86, at temperatures between 4° C. and 20° C., and these lower degradation values are already noticed after short durations. In other words, the inventive beta-lactamase substrate compounds degrade slower than the reference beta-lactamase substrate compounds, i.e. have a lower degradation, and are more stable.

Advantageously, β is recognizable by the carbapenemase OXA-40 and/or the carbapenemase OXA-58. In other words, β can be hydrolyzed by the carbapenemase OXA-40 and/or the carbapenemase OXA-58. Consequently, the beta-lactamase substrate compound can advantageously be used in the electrochemical detection of bacteria producing the carbapenemase OXA-40 and/or the carbapenemase OXA-58.

According to a second aspect of the present disclosure, there is provided an electrochemical method for determination of the presence of bacteria producing beta-lactamases in a sample as set out in the appended claims.

The method comprises the use of a beta-lactamase substrate compound according to the first aspect of the present disclosure. Advantageously, the method comprises the detection of the hydrolysis products of the beta-lactamase substrate compound, according to the present disclosure resulting from the hydrolysis of the beta-lactam ring by beta-lactamases.

The method comprises preparing a medium comprising a beta-lactamase substrate compound according to the first aspect of the present disclosure. The medium can be prepared by means of methods or techniques known in the art. A preferred medium is a succinate buffer (pH 5.5) comprising the beta-lactamase compound.

A sample to be analyzed is then incubated with the medium, thereby obtaining an incubated medium. Non-limiting examples of sample are biological samples, samples of environmental origin, food samples and culture mediums. The incubation is performed by methods or techniques known in the art. Advantageously, the incubation is performed at 37° C.

The incubation is carried out for a duration sufficiently long to allow the hydrolysis of the beta-lactam ring of the beta-lactamase substrate compound according to the present disclosure by the sample to be analyzed.

Advantageously, the incubation is performed for a duration of at least 10 minutes, such as at least 20 minutes, preferably at least 30 minutes.

Advantageously, the incubation is performed for a duration of at most 12 hours, preferably at most 8 hours, more preferably at most 4 hours, such as at most 3 hours, 2 hours or 1 hour.

The incubated medium is then subjected to cyclic voltammetry, thereby obtaining an intensity value of the anodic current of the incubated medium. A negative control, i.e. a medium substantially free from bacteria producing beta-lactamases and free from beta-lactamases, is subjected to cyclic voltammetry as well, thereby obtaining an intensity value of the anodic current of the negative control.

The cyclic voltammetry, also called amperometric detection, is performed by means of a working electrode. The working electrode comprises carbon (i.e. is carbon-based), a noble metal (i.e. is noble-metal based) and/or a metal oxide (i.e. is metal oxide-based).

Next, an oxidation current is obtained by measuring the difference of intensity value of the anodic current of the incubated medium and the negative control. An oxidation current higher than 0 nA is a measure for the presence of the hydrolysis products of the beta-lactamase substrate compound in the incubated medium, and thus of the presence of beta-lactamases and/or bacteria producing beta-lactamases in the sample.

Advantageously, preparing the medium comprises preparing the beta-lactamase substrate compound according to the present disclosure.

Advantageously, the preparing the beta-lactamase substrate compound according to the present disclosure comprises the steps of reacting a cephalosporin compound and a carboxylic acid comprising the functional group β by means of amide coupling, thereby obtaining a first intermediate compound.

The cephalosporin compound comprises a (6R,7R)-configuration. The first intermediate compound also comprises such a (6R,7R)-configuration. In other words, the (6R,7R)-configuration is maintained, i.e. preserved, in the first intermediate compound.

Advantageously, the cephalosporin compound comprises a diphenylmethyl ester group. Advantageously, the cephalosporin compound is according to formula (III):

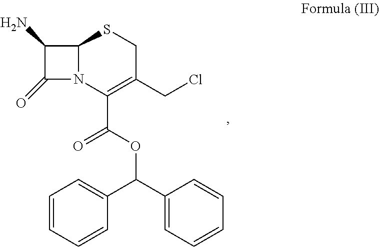 [SMILES: N[C@@H]1C(=O)N2C(C(=O)OC(c3ccccc3)c3ccccc3)=C(CCl)CS[C@H]12]

wherein the compound according to formula (III) comprises a (6R,7R)-configuration in the 4-atom ring structure comprising a nitrogen atom and three carbon atoms.

Advantageously, the carboxylic acid comprising the functional group β is formic acid, acetic acid, trifluoroacetic acid, or a compound according to formula (IV):

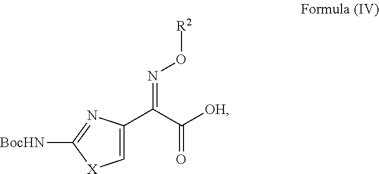

wherein
X is S or O, and
R² is C₁-C₁₆ alkyl, optionally comprising one or more carbonyl, hydroxyl, ether, ester or carboxyl functional groups.

Advantageously, R² is C₁-C₆ alkyl, optionally comprising one or more carbonyl, hydroxyl, ether, ester or carboxyl functional groups. Non-limiting examples of R² include methyl, ethyl, n-propyl, iso-propyl, —C(CH₃)₂COOH, and —C(CH₃)₂C(O)OC(CH₃)₃.

Advantageously, the first intermediate compound reacts with a compound comprising the functional group R by means of Wittig olefination, thereby obtaining a second intermediate compound.

Advantageously, the compound comprising the functional group R is selected from the group consisting of 4-nitrobenzaldehyde, 4-cyanobenzaldehyde, 4-sulfonebenzaldehyde, 2-nitro-4-cyanobenzaldehyde, 2,4-dicyanobenzaldehyde, 3,5-dinitrobenzaldehyde and 3,4-dinitrobenzaldehyde.

The second intermediate compound also comprises the (6R,7R)-configuration of the first intermediate compound, and thus of the cephalosporin compound. Advantageously, the first and the second intermediate compound comprise the diphenylmethyl ester group of the cephalosporin compound, i.e. the diphenylmethyl ester group is maintained, i.e. preserved, in the first and the second intermediate compound.

Advantageously, the second intermediate compound is then deprotected, thereby obtaining the beta-lactamase substrate compound. Advantageously, the deprotection of the second intermediate compound comprises dissolving the second intermediate compound in trifluoroacetic acid, thereby cleaving the diphenylmethyl ester group.

The Wittig olefination and the deprotection can be performed separately, or can be performed in a single step.

According to a third aspect of the present disclosure, there is provided a diagnostic kit for the detection of the presence of bacteria producing beta-lactamases in a sample as set out in the appended claims. The diagnostic kit comprises a beta-lactamase substrate compound according to the present disclosure.

An advantage of the beta-lactamase substrate compounds of the present disclosure, and in particular of the functionalized phenyl group (R in formula (I)) is an improved stability over time, up to at least 18 weeks, as compared to beta-lactamase substrate compounds for electrochemical detection known in the art, in particular HMRZ-86.

Another advantage of the beta-lactamase substrate compounds of the present disclosure, and in particular of the group β, is that a wider range of bacteria producing beta-lactamases can be detected, especially the beta-lactamase producing bacteria (or bacteria strains) CTXM, KPC/OXA-48, OXA-40, OXA-48, OXA-58, NDM, IMP-13, and/or VIM. Type(s)
as compared to beta-lactamase substrate compounds for electrochemical detection known in the art, in particular HMRZ-86. Without wishing to be bound by any theory, the inventors believe that this is realized by using β groups which are significantly smaller than the β group of for example HMRZ-86, in particular when β is hydrogen, methyl or perfluoromethyl.

### BRIEF DESCRIPTION OF THE DRAWINGS

Aspects of the present disclosure will now be described in more detail with reference to the appended drawings, wherein same reference numerals illustrate same features and wherein:

FIG. 1 represents the oxidation current measured for 4 concentrations of beta-lactamases for HMRZ-86 as reference beta-lactamase substrate compound and 7 beta-lactamase substrate compounds according to the present disclosure.

FIG. 2 represents the oxidation current measured for 4 concentrations of beta-lactamases for 3 beta-lactamase substrate compounds according to the present disclosure and having a single moiety at position 4 of the phenyl ring.

FIG. 3 represents the oxidation current measured for 4 concentrations of beta-lactamases for 3 beta-lactamase substrate compounds according to the present disclosure and having moieties at positions 2 and 4 of the phenyl ring.

FIG. 4 represents the cyclic voltammetry of the reference beta-lactamase substrate compound in the absence and in the presence of a beta-lactamase.

FIG. 5 represents the cyclic voltammetry of a beta-lactamase substrate compound of the present disclosure in the absence and in the presence of a beta-lactamase.

FIG. 6 represents the oxidation current measured for 4 concentrations of beta-lactamases for a beta-lactamase substrate compound according to the present disclosure at different concentrations and for HMRZ-86 as reference beta-lactamase substrate compound.

FIG. 7 represents the oxidation current for blood cultures comprising different beta-lactamase producing bacteria strains and a negative control for a beta-lactamase substrate compound according to the present disclosure and for HMRZ-86 as reference beta-lactamase substrate compound.

FIG. 8 represents the stability at 4° C. for up to 18 weeks for a beta-lactamase substrate compound according to the present disclosure and for HMRZ-86 as reference beta-lactamase substrate compound in relation to a DMF reference.

FIG. 9 represents the stability at 20° C. for up to 18 weeks for a beta-lactamase substrate compound according to the present disclosure and for HMRZ-86 as reference beta-lactamase substrate compound in relation to a DMF reference.

FIG. 10 represents the stability at 4° C. for up to 18 weeks for a beta-lactamase substrate compound according to the present disclosure and for HMRZ-86 as reference beta-lactamase substrate compound in relation to a succinate buffer reference.

FIG. 11 represents the stability at 20° C. for up to 18 weeks for a beta-lactamase substrate compound according to the present disclosure and for HMRZ-86 as reference beta-lactamase substrate compound in relation to a succinate buffer reference.

FIG. 12 represents the oxidation current for 3 further beta-lactamase substrate compounds of the present disclosure for the beta-lactamase KPC.

FIG. 13 represents the oxidation current for 4 different beta-lactamases for the 3 further beta-lactamase substrate compounds of the present disclosure and for HMRZ-86 as reference beta-lactamase substrate compound.

FIG. 14 represents the oxidation current for 3 different beta-lactamases and a negative control for a beta-lactamase substrate compound of the present disclosure and for HMRZ-86 as reference beta-lactamase substrate compound.

FIG. 15 represents the stability at 20° C. for up to 26 weeks for three beta-lactamase substrate compounds according to the present disclosure in relation to a dimethylformamide (DMF) reference.

### DETAILED DESCRIPTION

The beta-lactamase substrate compounds for electrochemical detection of beta-lactamases according to the present disclosure are hydrolysable by one or more beta-lactamases. Further, like the commercially available HMRZ-86 beta-lactamase substrate compound, they are electrically active, indicating that they can be used in the electrochemical detection of bacteria producing beta-lactamases. More precisely, upon hydrolysis of the beta-lactamase substrate compounds the hydrolysis products can be electrochemically detected. The presence of hydrolysis products of the beta-lactamase substrate compound indicates the presence of beta-lactamases, and thus of bacteria producing these.

The beta-lactamase substrate compound comprises a cephalosporin core to which are attached functional groups β and R. More particularly, the beta-lactamase substrate compound is according to formula (I)

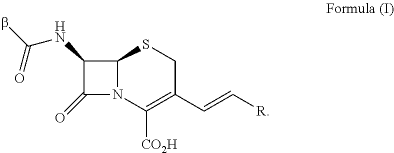

Advantageously, group β is a functional group which is recognizable by a beta-lactamase, advantageously by a plurality of beta-lactamases. In other words, the group β is involved in the selective recognition with beta-lactamases. Hence, the beta-lactamases which are capable to detect the functional group β are the beta-lactamases which will hydrolyze the beta-lactamase substrate compound. Consequently, the beta-lactamases which are capable to detect the functional group β are capable of being detected by electrochemical detection by means of the beta-lactamase substrate compound according to formula (I).

Advantageously, group β is hydrogen, C₁-C₆ alkyl, C₁-C₆ fluoroalkyl or is according to formula (VII):

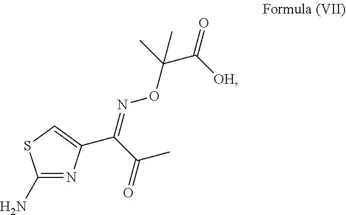 [SMILES: CC(=O)/C(=N\OC(C)(C)C(=O)O)c1csc(N)n1]

for example methyl (CH₃) or perfluoromethyl (CF₃).

Advantageously, group R is a functionalized phenyl functional group. The inventors have surprisingly discovered that the functionalized phenyl group contributes to the stability of the beta-lactamase substrate compound over time. Advantageously, R is selected from the group consisting of 4-cyanophenyl, 4-nitrophenyl, 4-sulfonephenyl, 2,4-dicyanophenyl, 2-nitro-4-cyanophenyl, 3,4-dinitrophenyl and 3,5-dinitrophenyl.

The beta-lactamase substrate compound is advantageously obtained, i.e. synthesized, by an amide coupling of a cephalosporin compound with a carboxylic acid comprising the functional group β, followed by a Wittig olefination with a compound comprising the functional group R, and the removal of all acid sensitive protecting groups with trifluoroacetic acid (TFA) (so-called deprotection).

Advantageously, the cephalosporin compound comprises a diphenylmethyl ester group. Advantageously, the cephalosporin compound is according to formula (III):

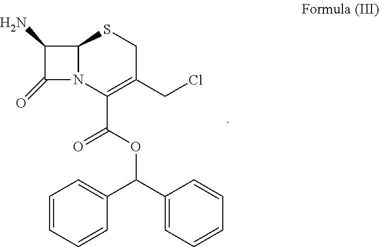 [SMILES: N[C@@H]1C(=O)N2C(C(=O)OC(c3ccccc3)c3ccccc3)=C(CCl)CS[C@H]12]

When β is according to formula (VII), the carboxylic acid is advantageously according to formula (VIII):

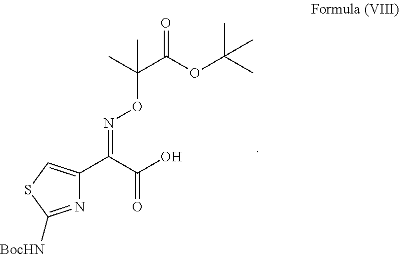 [SMILES: CC(C)(C)OC(=O)Nc1nc(/C(=N/OC(C)(C)C(=O)OC(C)(C)C)C(=O)O)cs1]

The carboxylic acid according to formula (VIII) can be obtained by (synthesis) methods known in the art.

When β is H, CH₃ or CF₃, the carboxylic acid is advantageously formic acid, acetic acid or trifluoroacetic acid.

The amide coupling of the cephalosporin compound with the carboxylic acid can be performed by methods known in the art. Advantageously, the amide coupling is performed using EDC.HCl in the presence of the catalyst 4-dimethylaminopyridine (DMAP).

The amide coupling of the cephalosporin compound of formula (III) with the carboxylic acid results in a first intermediate compound according to formula (V):

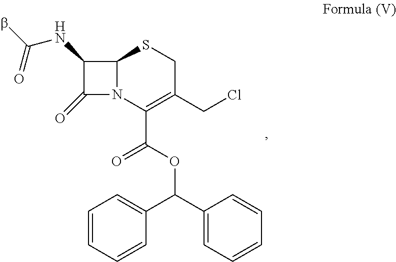

wherein β is as described hereinabove. The first intermediate compound comprises the diphenylmethyl ester group of the cephalosporin compound.

The first intermediate compound is then reacted with a compound comprising the functional group R in a Wittig olefination, thereby obtaining a second intermediate compound. The second intermediate compound comprises the diphenylmethyl ester group of the cephalosporin compound.

When R is 4-cyanophenyl, the compound comprising 4-cyanophenyl advantageously is 4-cyanobenzaldehyde. When R is 4-nitrophenyl, the compound comprising 4-nitrophenyl advantageously is 4-nitrobenzaldehyde. When R is 4-sulfonephenyl, the compound comprising 4-sulfonephenyl advantageously is 4-sulfonebenzaldehyde. When R is 2,4-dicyanophenyl, the compound comprising 2,4-dicyanophenyl advantageously is 2,4-dicyanobenzaldehyde. When R is 2-nitro-4-cyanophenyl, the compound comprising 2-nitro-4-cyanophenyl advantageously is 2-nitro-4-cyanobenzaldehyde. When R is 3,4-dinitrophenyl, the compound comprising 3,4-dinitrophenyl advantageously is 3,4-dinitrobenzaldehyde. When R is 3,5-dinitrophenyl, the compound comprising 3,5-dinitrophenyl advantageously is 3,5-dinitrobenzaldehyde.

Advantageously, the Wittig olefination is performed by methods known in the art, in particular at conditions known in the art.

Advantageously, the second intermediate compound is then filtered on silica gel chromatography.

The second intermediate compound is then deprotected, thereby obtaining the beta-lactamase substrate compound according to the present disclosure. Advantageously, deprotection of the second intermediate compound comprises dissolving the second intermediate compound in trifluoroacetic acid (TFA) to cleave all acid-sensitive protecting groups. Advantageously, semi-preparative RP-C18 HPLC purification is performed after cleavage of the acid-sensitive protecting groups.

The inventors have surprisingly discovered that the beta-lactamase substrate compounds according to the present disclosure where group β is according to formula (II) and group R is as defined hereinabove show a significant improvement of the stability of the substrate compound when compared to HMRZ-86. Particularly preferred beta-lactamase substrate compounds which show improved stability include substrate compounds where group β is according to formula (II) and group R is 2,4-dicyanophenyl, substrate compounds where group β is according to formula (II) and group R is 2-nitro-4-cyanophenyl.

Advantageously, the stability of the beta-lactamase substrate compound according to the present disclosure is expressed by the evolution of the percentage of degradation over time. The lower the (percentage of) degradation after a predetermined time, the better the stability of the beta-lactamase substrate compound.

Advantageously, in the present disclosure, the degradation is determined from the difference of the integration of nuclear magnetic resonance (NMR) measurement signals of the beta-lactamase substrate compound according to the present disclosure and of a reference. A particularly suitable reference is dimethylformamide (DMF).

More particularly, when the degradation is determined by means of NMR measurements, a first sample comprising the beta-lactamase substrate compound according to the present disclosure and a second sample comprising the reference, e.g. DMF, are heated or cooled to a predefined temperature, after which the NMR measurement is performed on both samples (measurement at time=0). The same measurement is repeated on the samples after a predefined time (time=x), while their temperature is kept constant at the predefined temperature from time=0 to time=x. The difference of the integration of NMR measurement signals of both samples is determined by comparing between time=0 and time=x, thereby allowing to define the degree (percentage) of degradation of the beta-lactamase substrate compound at time=x.

When the reference is DMF, and the stability is tested at 4° C., the beta-lactamase substrate compound according to the present disclosure advantageously has a degradation after 1 day of at most 20%, preferably at most 10%, more preferably at most 5%, such as below 1%, or even 0%.

When the reference is DMF, and the stability is tested at 4° C., the beta-lactamase substrate compound advantageously has a degradation after 1 week of at most 40%, preferably at most 30%, more preferably at most 20%, such as below 10%.

When the reference is DMF, and the stability is tested at 4° C., the beta-lactamase substrate compound according to the present disclosure advantageously has a degradation after 4 weeks of at most 50%, preferably at most 40%, more preferably at most 30%, such as below 20%.

When the reference is DMF, and the stability is tested at 4° C., the beta-lactamase substrate compound according to the present disclosure advantageously has a degradation after 18 weeks of at most 70%, preferably at most 65%, more preferably at most 60%, such as below 55%.

When the reference is DMF, and the stability is tested at 4° C., the beta-lactamase substrate compound according to the present disclosure advantageously has a degradation after 26 weeks of at most 70%, preferably at most 65%, more preferably at most 60%, such as below 55%.

When the reference is DMF and the stability is tested at 20° C., the beta-lactamase substrate compound according to the present disclosure has a degradation after 1 day of at most 40%, preferably at most 30%, more preferably at most 20%, such as below 10%.

When the reference is DMF and the stability is tested at 20° C., the beta-lactamase substrate compound according to the present disclosure has a degradation after 1 week of at most 60%, preferably at most 50%, more preferably at most 40%, such as below 30%.

When the reference is DMF and the stability is tested at 20° C., the beta-lactamase substrate compound according to the present disclosure has a degradation after 4 weeks of at most 80%, preferably at most 70%, more preferably at most 60%, such as at most 50%.

When the reference is DMF and the stability is tested at 20° C., the beta-lactamase substrate compound according to the present disclosure has a degradation after 18 weeks of at most 85%, preferably at most 80%, more preferably at most 75%, such as at most 70%, or at most 65%.

When the reference is DMF and the stability is tested at 20° C., the beta-lactamase substrate compound according to the present disclosure has a degradation after 26 weeks of at most 92%, preferably at most 90.

When the reference is a 22 mM succinate buffer, and the stability is tested at 4° C., the beta-lactamase substrate compound according to the present disclosure advantageously has a degradation after 1 week of at most 20%, preferably at most 10%, more preferably at most 5%, such as below 1%, or even 0%.

When the reference is a 22 mM succinate buffer, and the stability is tested at 4° C., the beta-lactamase substrate compound according to the present disclosure advantageously has a degradation after 2 weeks of at most 20%, preferably at most 10, and/or a degradation after 4 weeks of at most 30%, preferably at most 20%, and/or a degradation after 18 weeks of at most 50%, preferably at most 40%.

When the reference is a 22 mM succinate buffer, and the stability is tested at 4° C., the beta-lactamase substrate compound according to the present disclosure advantageously has a degradation after 1 week of at most 20%, preferably at most 10%, more preferably at most 5%, such as below 1%, or even 0%.

When the reference is a 22 mM succinate buffer, and the stability is tested at 4° C., the beta-lactamase substrate compound according to the present disclosure advantageously has a degradation after 2 weeks of at most 20%, preferably at most 10, and/or a degradation after 4 weeks of at most 30%, preferably at most 20%, and/or a degradation after 18 weeks of at most 50%, preferably at most 40%.

The inventors have also surprisingly discovered that the beta-lactamase substrate compounds according to the present disclosure where group β is H, C₁-C₁₆ alkyl, or C₁-C₁₆ fluoroalkyl and group R is 4-cyanophenyl are capable to electrochemically detect a wider range of beta-lactamases and bacteria producing beta-lactamases when compared to HMRZ-86. Particularly preferred beta-lactamase substrate compounds according to the present disclosure which can electrochemically detect a larger variety of beta-lactamases and bacteria producing beta-lactamases include substrates according to formula (VI)

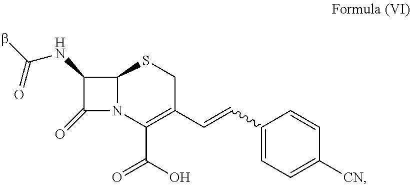

wherein β is H, CH₃ or CF₃, and group R is 4-cyanophenyl.

The electrochemical detection of a sample which has to be analyzed for the presence (or absence) of beta-lactamases and/or bacteria producing beta-lactamases comprises an amperometric measurement of an incubated medium. The amperometric measurement allows the detection of hydrolysis products of the beta-lactamase substrate compound. The presence of such hydrolysis products is an indication for the presence of beta-lactamases, and thus advantageously also of bacteria producing these beta-lactamases.

Advantageously, an incubated medium is obtained by methods known in the art. A preferred incubation method comprises incubating the sample to be analyzed with a medium comprising a beta-lactamase substrate compound for a sufficient duration to allow the beta-lactamase enzymes (if present) of the sample to hydrolyze the beta-lactamase substrate compound. Advantageously, the incubation duration is at most 12 hours, preferably at most 8 hours, more preferably at most 4 hours.

Advantageously, the amperometric measurement of the incubated medium is performed by methods known in the art. A particularly preferred method comprises cyclic voltammetry of the incubated medium by means of a working electrode based on carbon or based on a noble metal or based on metal oxide.

Advantageously, the same amperometric measurement is performed on a negative control in parallel, wherein the negative control is a sample devoid of any beta-lactamases and any bacteria producing beta-lactamases. The negative control allows to validate the measurement.

Advantageously, the amperometric measurement results in an intensity value of the anodic current for the sample to be analyzed and for the negative control. Advantageously, the intensity of the anodic current is measured in a potential range comprised between +0.1 V and +0.5 V vs. Ag/AgCl.

The difference between intensity values, the oxidation current, is a measure for the presence of hydrolysis products of the beta-lactamase substrate compound in the incubated medium, and thus of the presence of beta-lactamases or bacteria producing beta-lactamases in the analyzed sample.

Advantageously, the beta-lactamase substrate compounds according to the present disclosure allow the detection of beta-lactamase producing bacteria (or bacteria strains) CTXM, KPC/OXA-48, OXA-40, OXA-48, OXA-58, NDM, IMP-13, and VIM.

The present disclosure will be described in details in the following examples and figures presented as preferred and non-limiting embodiments of the present disclosure.

### EXAMPLES

### Example 1

7 beta-lactamase substrate compounds according to the present disclosure were produced. All 7 compounds comprised a group β according to formula (VII). Their group R was 2-nitro-4-cynanophenyl, 2,4-dicyanophenyl, 3,5-dinitrophenyl, 3,4-dinitrophenyl, 4-nitrophenyl, 4-cyanophenyl and 4-sulfonephenyl, respectively.

As reference, beta-lactamase substrate compound HMRZ-86 was provided. HMRZ-86 comprises a group β according to formula (VII) and 2,4-dinitrophenyl as group R.

A medium comprising 1.2 mM of substrate in a succinate buffer of pH 5.5 was prepared for each of the 8 beta-lactamase substrate compounds (7 inventive ones and the HMRZ-86 reference one).

4 different samples to be analyzed were prepared, each comprising KPC as beta-lactamase in a standard 2,4,6-tri-2-pyridinyl-1,3,5-triazine (TPTZ) buffer but in different concentrations: 239.4, 59.85, 9.975 and 4.985 ng/ml (ng of KPC per mL of TPTZ buffer). The two first concentrations of KPC aim to determine the capacity of the derivative to detect the presence of beta-lactamase whereas the two lowest concentrations aim to address the limit of detection.

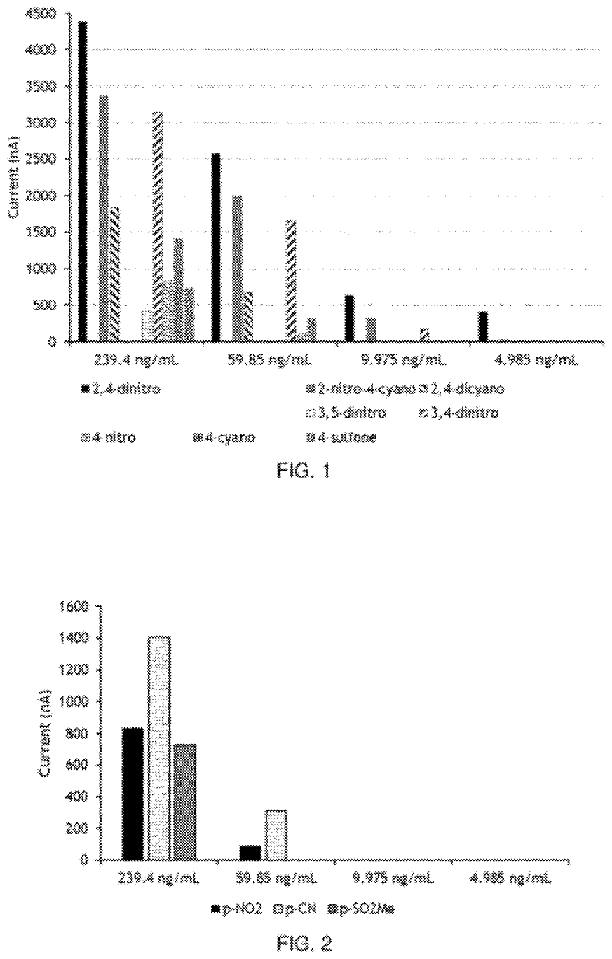

Each of the 8 samples was added to each of the 4 mediums (in other words 32 combinations) and incubated for 30 minutes at 37° C. Next, an amperometric measurement was performed on all 32 of the incubated mediums, and the oxidation current was determined. The device used was a Metrohm Dropsens and the electrodes were Metrohm Dropsens SCPE 110 electrodes. The oxidation current is the maximum current value measured between 0.1 V and 0.5 V vs. Ag/AgCl. FIG. 1 shows the oxidation currents for all 32 incubated mediums, where “2,4-dinitro” refers to the HMRZ-86 substrate compound, “2-nitro-4-cyano” refers to the substrate compound comprising 2-nitro-4-cyanophenyl as group R, etc.

It is clear that HMRZ-86 allowed the detection of the beta-lactamases at all 4 concentrations. Further, substrate compounds with as R group 2,4-dinitrophenyl (reference HMRZ-86) or 3,4-dinitrophenyl could detect the beta-lactamase concentration of 9.975 ng/mL, whereas with R group 3,5-dinitrophenyl this was not the case.

FIG. 2 shows the results for only the beta-lactamase substrate compounds having R groups with a single moiety at the para-position: 4-nitrophenyl (p-NO2), 4-cyanophenyl (p-CN) and 4-sulfonephenyl (p-SO2Me). It is clear that the highest oxidation currents were measured for the substrate compound with 4-cyanophenyl as R, whereas for R being 4-sulfonephenyl only the highest beta-lactamase concentration was detected.

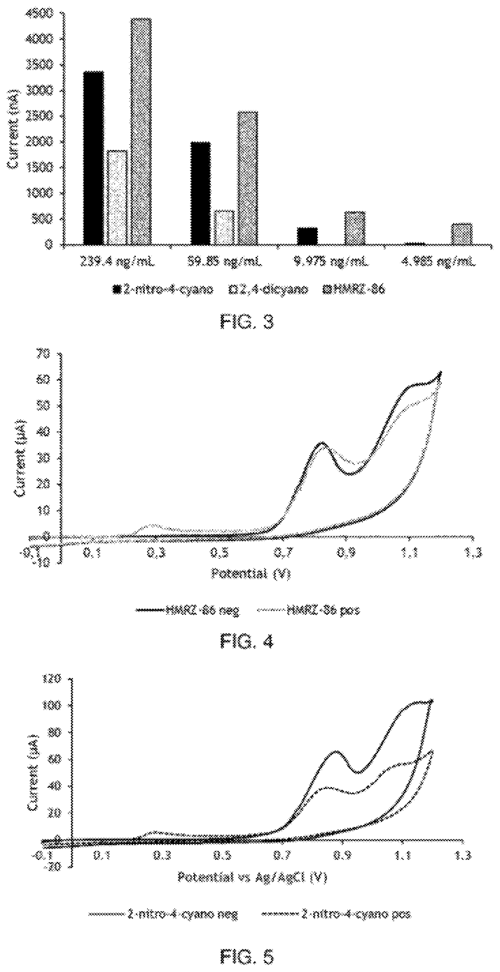

FIG. 3 shows the results for only the beta-lactamase substrate compounds having R groups with two moieties, one at position 2 and one at position 4: the reference 2,4-dinitrophenyl (HMRZ-86), as well as 2-nitro-4-cyanophenyl(2-nitro-4-cyano) and 2,4-dicyanophenyl (2,4-dicyano). It is clear from FIG. 3 that a nitro moiety at position 2 allowed to detect the beta-lactamases at all 4 concentrations, whereas with a cyano moiety at position 2 only the 2 highest concentrations were detected.

To confirm that the measured oxidation current is coming from the detection of beta-lactamases (or their hydrolysis products), cyclic voltammetry was performed on the HMRZ-86 reference substrate compound, and on the beta-lactamase substrate compound with R being 2-nitro-4-cyanophenyl. Cyclic voltammetry was performed with the same equipment as used for the amperometric measurement, and 1 entire voltage cycle was executed, and was performed in the presence (positive) and in the absence (negative) of beta-lactamases in the sample.

FIG. 4 shows the result for the HMRZ-86 reference (with R being 2,4-dinitrophenyl), and FIG. 5 shows the result for the beta-lactamase substrate compound (“2-nitro-4-cyano”) according to the present disclosure, wherein “neg” stands for the sample without beta-lactamases and “pos” for the sample with beta-lactamases. For both beta-lactamase substrate compounds, an oxidation signal specific for the presence of beta-lactamases (more particularly the hydrolysis products of the substrate compound after hydrolysis by the beta-lactamases) is present at a potential of +0.3 V vs. Ag/AgCl, since this signal is only present for the “positive” samples. The two other peaks are present for both “positive” and “negative” samples and are thus not specific for the presence of beta-lactamases. More particularly, the peak at +0.8 V vs. Ag/AgCl is related to buffer oxidation and the peak at +1.0 V vs. Ag/AgCl is related to sulphur oxidation.

### Example 2

To test the impact of the concentration of the beta-lactamase substrate compound on the possibility to detect beta-lactamases, a further medium comprising 2.4 mM of the beta-lactamase substrate compound with R being 2-nitro-4-cyanophenyl was prepared. This concentration was compared to the 1.2 mM medium with the same beta-lactamase substrate compound of example 1, and to the 1.2 mM reference medium (HMRZ-86). The same four beta-lactamase concentrations as in Example 1 were tested (i.e. the same samples). The incubation and measurement were performed as in Example 1.

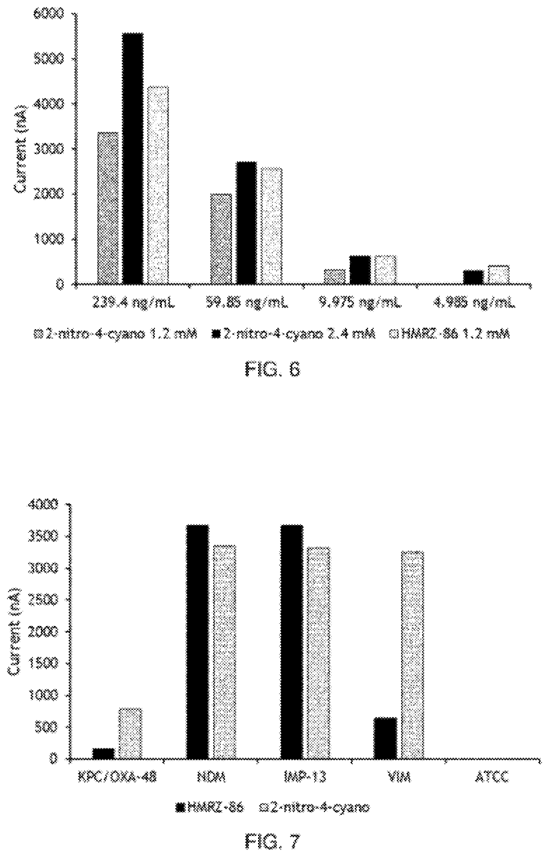

FIG. 6 shows the results. It is clear that increasing the concentration of the beta-lactamase substrate compound (2-nitro-4-cyano) according to the present disclosure significantly increases the oxidation current, resulting in a more efficient detection of the beta-lactamases at the lowest concentrations thereof. Further, the 2.4 mM concentration of the beta-lactamase substrate compound according to the present disclosure showed oxidations currents in line with these of the 1.2 mM HMRZ-86 reference (2,4-dinitro), and even being higher than the reference for the 2 highest beta-lactamase concentrations.

### Example 3

The possibility of the beta-lactamase substrate compounds to detect bacteria strains producing different beta-lactamases was tested for the reference HMRZ-86 of example 1 (at 1.2 mM) and the beta-lactamase substrate compound with R being 2-nitro-4-cyano of Example 1 (at 2.4 mM). 4 blood cultures comprising beta-lactamase producing bacteria strains that produce KPC/OXA-48, NDM, IMP-13 and VIM type, respectively, were prepared, as well as a negative control with ATCC, which is a bacteria strain that does not produce any beta-lactamase. Consequently, amperometric voltammetry of the negative control with ATCC should give an oxidation current of 0 nA for the measurement to be valid. The blood cultures did not necessarily comprise the same concentration of each beta-lactamase producing bacteria strain, so the measurement results are qualitative (is the bacteria strain detected or not) but not quantitative (not possible to say whether certain bacteria strains can be detected more easily).

FIG. 7 shows the results, wherein the oxidation current for the negative control (ATCC) was 0 nA, indicating a valid measurement. All beta-lactamase producing bacteria strains were detected by both beta-lactamase substrate compounds. However, for KPC/OXA-48 and VIM, the oxidation current of the substrate compound of the present disclosure (“2-nitro-4-cyano”) was significantly higher.

### Example 4

The stability of the beta-lactamase substrate compounds over time at different temperatures was tested the reference HMRZ-86 of example 1 (at 1.2 mM) and the beta-lactamase substrate compound with R being 2-nitro-4-cyano of Example 1 (at 2.4 mM).

The stability was tested by a NMR stability study. The degradation was measured by measuring the evolution of the integration of NMR measurement signals of the molecule in relation to a dimethylformamide (DMF) reference. The stability of the beta-lactamase substrate compound is expressed by the percentage of degradation in function of time.

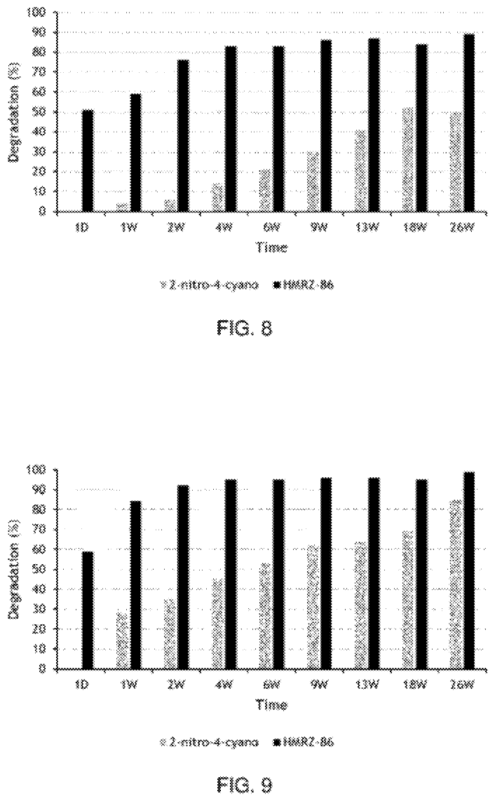

FIG. 8 shows the results at 4° C. for both beta-lactamase substrate compounds, wherein “HMRZ-86” refers to the reference and “2-nitro-4-cyano” to the inventive substrate compound. Measurements were performed after 1 day, 1 week, 2 weeks, up to 18 weeks. Whereas the HMRZ-86 reference already shows significant degradation of over 50% after 1 day, the substrate compound according to the present disclosure shows slight degradation (4%) only after 1 week. Further, the degradation of the substrate compound according to the present disclosure after 18 weeks is similar to the degradation of the reference after only 1 day. A clear improvement of the stability was thus noticed.

FIG. 9 shows the results at 20° C. for both beta-lactamase substrate compounds, wherein “HMRZ-86” refers to the reference and “2-nitro-4-cyano” to the inventive substrate compound. Measurements were performed after 1 day, 1 week, 2 weeks, up to 18 weeks. Whereas the HMRZ-86 reference already shows significant degradation of almost 60% after 1 day, the inventive substrate compound shows degradation (28%) starting only after 1 week. After 18 weeks, the reference has a degradation of over 90%, i.e. almost total degradation, whereas the inventive substrate compound still has a degradation below 70%.

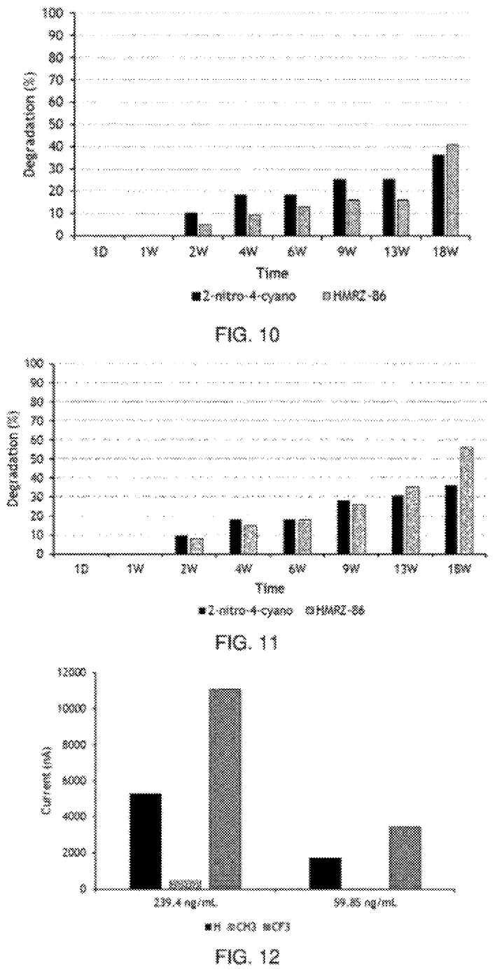

The stability of both beta-lactamase substrate compounds was also tested in relation to a 22 mM succinate buffer reference. FIG. 10 shows the results at 4° C. and FIG. 11 shows the results at 20° C., wherein “HMRZ-86” refers to the reference and “2-nitro-4-cyano” to the inventive substrate compound. Contrary to the dimethylformamide (DMF) reference, the degradation in relation to the succinate buffer was similar for both substrate compounds, with the first degradation starting after 2 weeks at both temperatures.

### Example 5

Three further beta-lactamase substrate compounds according to the present disclosure were produced. All three compounds comprised a group R being 4-cyanophenyl. Their group β was hydrogen (H), methyl (CH₃) and trifluoromethyl (CF₃) respectively.

A medium comprising 6.0 mM of each substrate in a succinate buffer of pH 5.5 was prepared. 2 different samples to be analyzed were prepared, each comprising KPC as beta-lactamase type in a standard 2,4,6-tri-2-pyridinyl-1,3,5-triazine (TPTZ) buffer, but in different concentrations: 239.4 ng/ml and 59.85 ng/ml, respectively (ng of KPC per mL of TPTZ buffer). Each of the 3 samples was added to each of the 2 mediums (in other words 6 combinations) and incubated for 30 minutes at 37° C. Next, an amperometric measurement was performed on all 6 of the incubated mediums, and the oxidation current was determined. The equipment used was the same as that of Example 1.

FIG. 12 shows the oxidation currents for all 6 incubated mediums, where “H”, “CH₃” and “CF₃” refer to the respective groups β. Whereas all 3 beta-lactamase substrate compounds are capable to detect the highest concentration of KPC, only the “H” and “CF₃” groups are capable to detect the lowest tested concentration. The inventors believe that this is related to a different affinity for the KPC beta-lactamase type, which can influence the speed of the hydrolysis. Consequently, the “CH₃” group comprising beta-lactamase substrate compound require a longer incubation time.

The possibility to detect different beta-lactamase type producing bacteria strains was also tested. As reference, a reference medium comprising 1.2 mM beta-lactamase substrate compound HMRZ-86 was prepared as well. HMRZ-86 comprises a group β according to formula (II) and 2,4-dinitrophenyl as group R. Samples comprising CTXM, OXA-40, OXA-48 and OXA-58 types were tested, as well as a negative control with ATCC, which is a bacteria strain that does not produce any beta-lactamase. The samples cultures did not necessarily comprise the same concentration of CTXM, OXA-40, OXA-48 and OXA-58 types, respectively, so the measurement results are qualitative (detection/no detection) but not quantitative (not possible to say which one can be detected more easily).

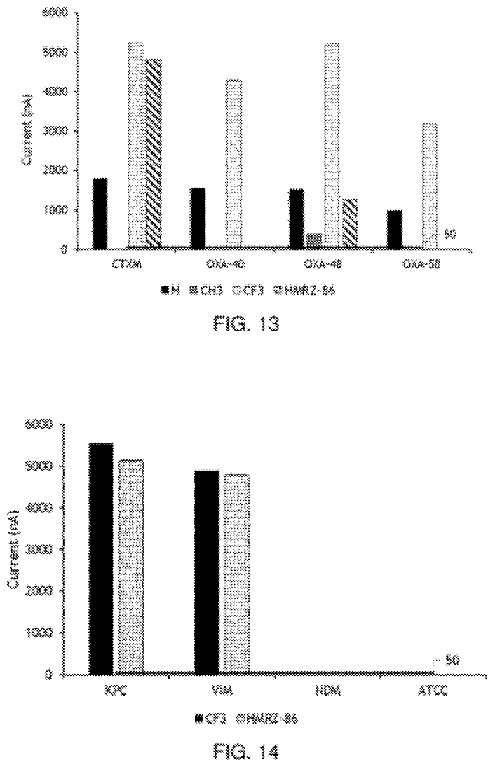

FIG. 13 shows that the reference HMRZ-86 is only capable of detecting the beta-lactamases CTXM type and OXA-48 type, and does not detect the carbapenemases OXA-40 type and OXA-58 type, whereas the beta-lactamase substrate compounds according to the present disclosure with group β being H and CF₃ are capable of detecting all 4 beta-lactamases. The negative control is not shown, but all values measured with it were 0 nA, indicating valid measurements However, the compound according to the present disclosure with group β being CH₃ detect OXA-48 type only.

The beta-lactamase with group β being CF₃ (6.0 mM), as well as the reference HMRZ-86 (1.2 mM) were also tested with blood cultures comprising bacteria strains producing the carbapenemase KPC type, the metallo-beta-lactamase VIM type and the metallo-beta-lactamase NDM type, as well as a blood culture comprising no bacteria strains producing beta-lactamases (negative control ATCC). FIG. 14 shows that both beta-lactamase substrate compounds are capable of detecting KPC type and VIM, type, but do not detect NDM type.

The negative control ATCC had an oxidation current of 0 nA, indicating that the measurements were valid.

The stability of all 3 beta-lactamase substrate compounds (R being 4-cyanophenyl, group β being H, CH₃ and CF₃, respectively), as well as the beta-lactamase having the same group R and a group β according to formula II was tested at 20° C. in DMF as reference. The beta-lactamase substrate compound with R being 4-cyanophenyl and β according to formula II showed no signs of degradation up to 26 weeks, indicating an excellent stability.

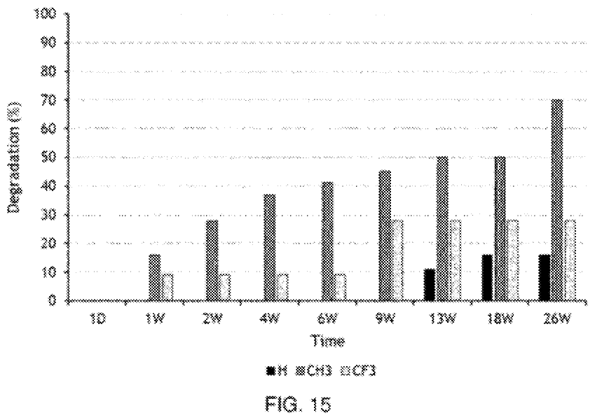

FIG. 15 shows the results for the other 3 beta-lactamase substrate compounds. The first signs of degradation are visible after 1 week (reference HMRZ-86 showed already almost 60% degradation after 1 week in the same conditions, see FIG. 9) for β being methyl and trifluoromethyl, whereas for β being hydrogen the first degradation is only noticed after 13 weeks.

### Example 6

The reactivities of HMRZ-86 and the CF₃ (β)/4-cyanophenyl (R) [formula (I)] compounds for enzymes from Ambler classes A (ESBLs and carbapenemases), B (metallo-beta-lactamases) and D (carbapenemases) were analyzed with recombinant enzymes.

A medium comprising 2.4 mM of the substrate compound with β being CF₃ and R being 4-cyanophenyl was prepared in a succinate buffer of pH 5.5. This medium was compared to the 1.2 mM reference medium (HMRZ-86) also prepared in a succinate buffer of pH 5.5. 30 different enzymes (8 from class A, 14 from class B and 8 from class D) were diluted in a standard TPTZ buffer. Each of the 30 samples were added to each of the two mediums and incubated for 10 minutes at 37° C. The measurements were performed as in Example 1.

Table 1 represents the comparison of reactivity between the HMRZ-86 and the CF₃ (β)/4-cyanophenyl (R) [formula (I)] compounds for enzymes (recombinant) from Ambler classes A (ESBLs and carbapenemases), B (metallo-beta-lactamases) and D (carbapenemases) antibiotics.

Table 1 shows the results, wherein the obtained reactivity of the CF₃/4-cyanophenyl compound is greater than the one from the HMRZ-86 on the class D carbapenemases with all the 8 variants of OXA types detected (only OXA-78, OXA-143 and OXA-163 types were detected with the HMRZ-86). Only the KPC-31 type variant was not detected with the CF3/4-cyanophenyl compound, but with the HMRZ-86).

TABLE 1

| Ambler | | | | Compound | |
|---|---|---|---|---|---|
| class | Enzymatic class | Enzyme | Sample | HMRZ-86 | CF₃/4-cyanostyryl |
| A | ESBL | CTX-M-2 | Protein | Detected | Detected |
| | | CTX-M-8 | Protein | Detected | Detected |
| | | CTX-M-14 | Protein | Detected | Detected |
| | | CTX-M-15 | Protein | Detected | Detected |
| | | CTX-M-25 | Protein | Detected | Detected |
| | Carbapenemase | KPC-3 | Protein | Detected | Detected |
| | | KPC-31 | Protein | Detected | Not detected |
| | | KPC-33 | Protein | Detected | Detected |
| B | Metallo-Beta-Lactamase | IMP-1 | Protein | Detected | Detected |
| | | IMP-3 | Protein | Detected | Detected |
| | | IMP-4 | Protein | Detected | Detected |
| | | IMP-11 | Protein | Detected | Detected |
| | | IMP-14 | Protein | Detected | Detected |
| | | IMP-16 | Protein | Detected | Detected |
| | | IMP-18 | Protein | Detected | Detected |
| | | IMP-22 | Protein | Detected | Detected |
| | | IMP-31 | Protein | Detected | Detected |
| | | IMP-61 | Protein | Detected | Detected |
| | | NDM-1 | Protein | Detected | Detected |
| | | VIM-5 | Protein | Detected | Detected |
| | | VIM-11 | Protein | Detected | Detected |
| | | VIM-13 | Protein | Detected | Detected |
| D | Carbapenemase | OXA-23 | Protein | Not detected | Detected |
| | | OXA-40 | Protein | Not detected | Detected |
| | | OXA-48 | Protein | Detected | Detected |
| | | OXA-58 | Protein | Not detected | Detected |
| | | OXA-51 | Protein | Not detected | Detected |
| | | OXA-143 | Protein | Detected | Detected |
| | | OXA-163 | Protein | Detected | Detected |
| | | OXA-213 | Protein | Not detected | Detected |

### Example 7

The reactivities of HMRZ-86 and the CF₃ (β)/4-cyanophenyl (R) [formula (I)] compounds for enzymes from Ambler classes C (AmpC cephalosporinases) were analyzed with AmpC positive clinical strains previously characterized by sequencing. These strains correspond to 11 different species, 24 different enzymes and 2 mechanisms of enzyme production [HCASE (hyperproductive) and CASEP (plasmid mediated (See Table 2) Table 2 represents the list of AmpC (Ambler class C cephalosporinases) positive clinical strains tested with the HMRZ-86 and the CF₃ (β)/4-cyanophenyl (R) [formula (I)] compounds.

TABLE 2

| Specie | Enzyme | Mechanism |
|---|---|---|
| Citrobacter freundii | CMY-48-like | HCASE |
| Enterobacter cloacae | ACC-1 | CASEP |
| Enterobacter cloacae complex | ACT-C42 | HCASE |
| Enterobacter cloacae complex | ACT-C99-like | HCASE |
| Enterobacter cloacae complex | ACT-17 | HCASE |
| Enterobacter cloacae complex | ACT-59-like | HCASE |
| Enterobacter cloacae complex | ACT-C81-like | HCASE |
| Enterobacter cloacae complex | ACT-77-like | HCASE |
| Enterobacter cloacae complex | ACT-C127-like | HCASE |
| Escherichia coli | CMY-42 | CASEP |
| Escherichia coli | DHA-1 | CASEP |
| Escherichia coli | CMY-4 | CASEP |
| Escherichia coli | ACC-1 | CASEP |
| Escherichia coli | CMY-2 | CASEP |
| Escherichia coli | ESC-77 | HCASE |
| Escherichia coli | ESC-144-like | HCASE |
| Escherichia coli | AmpC | HCASE |
| Escherichia coli | ESC-35-like | HCASE |
| Hafnia alvei | ACC-1b-like | HCASE |
| Hafnia alvei | AAC-1a | HCASE |
| Hafnia paralvei | ACC-1a | HCASE |
| Klebsiella aerogenes | AmpC | HCASE |
| Klebsiella pneumoniae | DHA-1 | CASEP |
| Klebsiella pneumoniae | DHA-7 | CASEP |
| Morganella morganii | DHA-13 | HCASE |
| Morganella morganii | DHA-1 | HCASE |
| Proteus mirabilis | DHA-1 | CASEP |
| Proteus mirabilis | CMY-2 | CASEP |
| Providencia stuartii | PSTU-2-like | HCASE |
| Providencia stuartii | PSTU-2 | HCASE |

The tested mediums were substrate compounds prepared in a succinate buffer of pH 5.5. The concentration analyzed were 2.4 mM for the compound with β being CF₃ and R being 4-cyanophenyl and 1.2 mM for the reference compound (HMRZ-86). Colonies from 30 AmpC positive clinical strains collected from solid culture medium were diluted in a standard TPTZ buffer. Each of the 30 samples were added to each of the two mediums and incubated for 10 minutes at 37° C. The measurements were performed as in Example 1. A negative clinical strain and an positive one (CTX-M) were analyzed as positive and negative controls.

All of the AmpC positive clinical strains were detected as positive with the CF₃/4-cyanophenyl compound, but only 53.3% ( 16/30) with the HMRZ-86.

1. A beta-lactamase substrate compound for electrochemical detection of beta-lactamases, wherein the beta-lactamase substrate compound is according to formula (I) 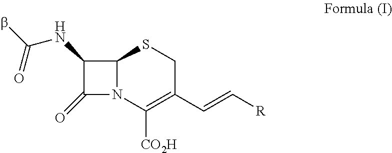
wherein
β is hydrogen, C₁-C₁₆ alkyl, C₁-C₁₆ fluoroalkyl or is according to formula (II): 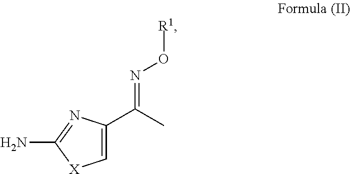
wherein
X is S or O, and
R¹ is C₁-C₁₆ alkyl, optionally comprising one or more carbonyl, hydroxyl, ether, ester or carboxyl functional groups; and
R is selected from the group consisting of 4-cyanophenyl, 4-nitrophenyl, 4-sulfonephenyl, 2,4-dicyanophenyl, 2-nitro-4-cyanophenyl, 3,4-dinitrophenyl and 3,5-dinitrophenyl;
wherein the beta-lactamase substrate compound is hydrolysable by a beta-lactamase; and
wherein the beta-lactamase substrate compound has a degradation at 4° C. equal to or lower than 50% after 4 weeks and equal to or lower than 70% after 18 weeks, and/or a degradation at 20° C. equal to or lower than 80% after 4 weeks and equal to or lower than 85% after 18 weeks, wherein the degradation is determined from a difference of an integration of nuclear magnetic resonance (NMR) measurement signals of the beta-lactamase substrate compound and of dimethylformamide.

2. The Beta-lactamase substrate compound according to claim 1, wherein β is according to formula (II), wherein X is S and R¹ is —C(CH₃)₂COOH, and R is 2,4-dicyanophenyl or 2-nitro-4-cyanophenyl.

3. The Beta-lactamase substrate compound according to claim 1, wherein β is hydrogen, methyl or perfluoromethyl and R is 4-cyanophenyl.

4. The Beta-lactamase substrate compound according to claim 1, having a degradation at 4° C. equal to or lower than 30% after 4 weeks and equal to or lower than 60% after 18 weeks.

5. The Beta-lactamase substrate compound according to claim 1, having a degradation at 20° C. equal to or lower than 70% after 4 weeks and equal to or lower than 80% after 18 weeks.

6. The Beta-lactamase substrate compound according to claim 1, wherein β is recognizable by OXA-40 type and/or OXA-58 type.

7. An electrochemical detection method for determination of a presence of bacteria producing beta-lactamases in a sample, the method comprising:
preparing a medium comprising a beta-lactamase substrate compound according to claim 1,
incubating a sample to be analyzed with the medium for a duration sufficiently long to allow hydrolysis of a beta-lactam ring of the beta-lactamase substrate compound by the sample to be analyzed, thereby obtaining an incubated medium,
performing cyclic voltammetry of the incubated medium and a negative control, thereby obtaining an intensity value of an anodic current of the incubated medium and the negative control, and
measuring the difference of intensity value of the anodic current of the incubated medium and the negative control, thereby obtaining an oxidation current,
wherein the cyclic voltammetry is performed by means of a working electrode, wherein the working electrode comprises carbon, a noble metal and/or a metal oxide, and
wherein an oxidation current higher than 0 nA indicates the presence of bacteria producing beta-lactamases in the sample.

8. The electrochemical detection method according to claim 7, wherein the sample is incubated with the medium for a duration of at least 30 minutes and at most 4 hours.

9. A method for obtaining the beta-lactamase substrate compound according to claim 1, the method comprising:
reacting a cephalosporin compound and a carboxylic acid comprising the functional group β by means of amide coupling, thereby obtaining a first intermediate compound,
reacting the intermediate compound with a compound comprising the functional group R by means of Wittig olefination, thereby obtaining a second intermediate compound, and
deprotecting the second intermediate compound, thereby obtaining the beta-lactamase substrate compound.

10. The method according to claim 9, wherein the cephalosporin compound comprises a diphenylmethyl ester group and is according to formula (III): 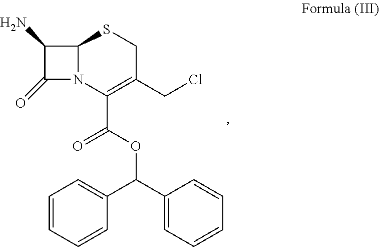 [SMILES: N[C@@H]1C(=O)N2C(C(=O)OC(c3ccccc3)c3ccccc3)=C(CCl)CS[C@H]12]
and wherein the first and the second intermediate compound comprise the diphenylmethyl ester group of the cephalosporin compound.

11. The method according to claim 9, wherein the deprotection of the second intermediate compound comprises dissolving the second intermediate compound in trifluoroacetic acid, thereby cleaving the diphenylmethyl ester group.

12. The method according to claim 9, wherein the carboxylic acid comprising the functional group β is formic acid, acetic acid, trifluoroacetic acid, or a compound according to formula (IV): 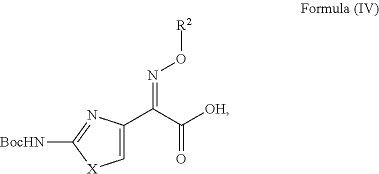
wherein
X is S or O, and
R² is C₁-C₁₆ alkyl, optionally comprising one or more carbonyl, hydroxyl, ether, ester or carboxyl functional groups.

13. The method according to claim 9, wherein the compound comprising the functional group R is selected from the group consisting of: 4-nitrobenzaldehyde, 4-cyanobenzaldehyde, 4-sulfonebenzaldehyde, 2-nitro-4-cyanobenzaldehyde, 2,4-dicyanobenzaldehyde, 3,4-dinitrobenzaldehyde and 3,5-dinitrobenzaldehyde.

14. The method according to claim 9, wherein the Wittig olefination and the deprotection are performed in a single step.

15. A diagnostic kit for the detection of the presence of bacteria producing beta-lactamases in a sample, comprising the beta-lactamase substrate compound according to claim 1.

16. A diagnostic kit for the detection of the presence of bacteria producing beta-lactamases in a sample, comprising the beta-lactamase substrate compound obtained by the method according to claim 9.
